(19) United States
(12) Patent Application Publication
(10) Pub. No.: US 2007/0008407 A1
(43) Pub. Date: Jan. 11, 2007

(54) IMAGE CAPTURING DEVICE FOR ELECTRONIC ENDOSCOPE

(75) Inventors: Kazuyuki YAMAMOTO, Tokyo (JP); Takayuki OGINO, Tokyo (JP); Akihiro ITO, Tokyo (JP); Seiichiro OKAMURA, Ibaraki (JP); Tomokazu YAMASHITA, Ibaraki (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/456,288

(22) Filed: Jul. 10, 2006

(30) Foreign Application Priority Data
Jul. 11, 2005 (JP) 2005-201113

(51) Int. Cl. H04N 7/18; A61B 1/00

(52) U.S. Cl. 348/65; 600/101

(57) ABSTRACT

An image capturing device for an electronic endoscope is provided with a solid-state image capturing element, an image capturing element holding frame, an objective lens barrel containing an objective optical system, an air-tight connection tube, a front end portion of the connection tube being air-tightly connected to the objective lens barrel, a rear end portion of the connection tube being air-tightly fitted on the image capturing element holding frame. Multiple connection lands to which multiple signal lines of a signal cable are connected are arranged on an outer circumferential surface, at a rear portion, of the image capturing element holding frame, the rear end portion of the connection tube being air-tightly connected to the outer circumference of the image capturing element holding frame on an front side, a size of an outline of the multiple signal lines being smaller than an outer shape of the connection tube.

### BACKGROUND OF THE INVENTION

The present invention relates to an image capturing device built in an distal end of an inserting section of an electronic endoscope.

Generally, the image capturing device for the electronic endoscope is configured such that a solid-state image capturing element is held by a holding frame. The outer circumferential surface of the holding frame is air-tightly adhered to a rear portion of an inner surface of a connecting tube, which is air-tightly connected to an objective lens barrel at the tip portion thereof. With such a configuration, when the endoscope is placed in a high-temperature high-pressure steam sterilizer, no steam or water enters portion where the solid-state image capturing element is implemented. An example of such a configuration is disclosed in Japanese Patent Provisional Publication No. P2000-60793A.

FIG. 7 shows an example of a conventional image capturing device for the electronic endoscope. In this example, a solid-state image capturing element 91 is held by an image capturing element holding frame 92, which is air-tightly surrounded by an air-tight connecting tube 93. Multiple signal lines 94 of a signal cable are connected to multiple connection lands 95, respectively, which are arranged on an outer circumferential surface of the connection tube 93 at a rear portion thereof.

The connection lands 95 are arranged on the outer surface (entire circumference) of the connection tube 93 at the rear portion as shown in FIG. 8. In FIG. 7, 96 denotes an objective lens barrel that holds an objective optical system 97 therein.

According to the conventional configuration described above, however, an outer length D of the multiple signal lines 94 connected to the connection lands 95 is relatively large. Since the inserting section of the endoscope is required to be as small as possible, the conventional configuration obstacles downsizing of the tip of the inserting section of the electronic endoscope. That is, the above configuration cannot be applied to a very small diameter electronic endoscope, or other components such as an illumination light guide or channel tube which should be made as thin as possible. Such a modification may deteriorate the function of the endoscope.

### SUMMARY OF THE INVENTION

The aspects of the present invention provide an image capturing device for an electronic endoscope which device is configured to prevent invasion of steam or water when processed by the high-temperature high-pressure steam sterilizer, and the outer size of the multiple signal lines connected to multiple connection lands, thereby contributing to downsizing and/or improving in performance of the electronic endoscope.

According to aspects of the invention, there is provided an image capturing device for an electronic endoscope, which is provided with a solid-state image capturing element, an image capturing element holding frame, an objective lens barrel containing an objective optical system, an air-tight connection tube, a front end portion of the connection tube being air-tightly connected to the objective lens barrel, a rear end portion of the connection tube being air-tightly fitted on the image capturing element holding frame. Multiple connection lands to which multiple signal lines of a signal cable are connected are arranged on an outer circumferential surface, at a rear portion, of the image capturing element holding frame, the rear end portion of the connection tube being air-tightly connected to the outer circumference of the image capturing element holding frame on an front side with respect to the rear portion where the connection lands are arranged, a size of an outline of the multiple signal lines being smaller than an outer shape of the connection tube.

An outer shape of the image capturing element holding frame at the rear portion where the multiple connection lands are arranged is formed smaller than an outer shape of the front portion where the air-tight connection tube is secured.

The image capturing device further includes a conductive shielding tube configured to surround the multiple connection lands, an outer shape of the shielding tube in an axial direction thereof is smaller than an outer shape of the objective lens barrel in the axial direction of the shielding tube, a tip end portion of the shielding tube being fitted on an outer circumference of the air-tight connection tube.

### BRIEF DESCRIPTION OF THE ACCOMPANYING DRAWINGS

FIG. 1 is a cross-sectional side view of an image capturing device according to a first embodiment of the invention.

FIG. 2 is a cross-sectional view of the image capturing device taken along line II-II of FIG. 1.

FIG. 3 is a cross-sectional side view of an inserting section of an electronic endoscope according to the first embodiment.

FIG. 4 is a cross-sectional side view of an image capturing device according to a second embodiment of the invention.

FIG. 5 is a partial perspective view of the image capturing device according to the second embodiment of the invention.

FIG. 6 is a cross-sectional side view of an image capturing device according to a third embodiment of the invention.

FIG. 7 is a cross-sectional side view showing the conventional image capturing unit incorporated in an objective optical system unit.

FIG. 8 is a partial perspective view of the conventional image capturing device.

### DETAILED DESCRIPTION OF THE EMBODIMENTS

Hereinafter, referring to the accompanying drawings, image capturing device according to embodiments of the invention will be described.

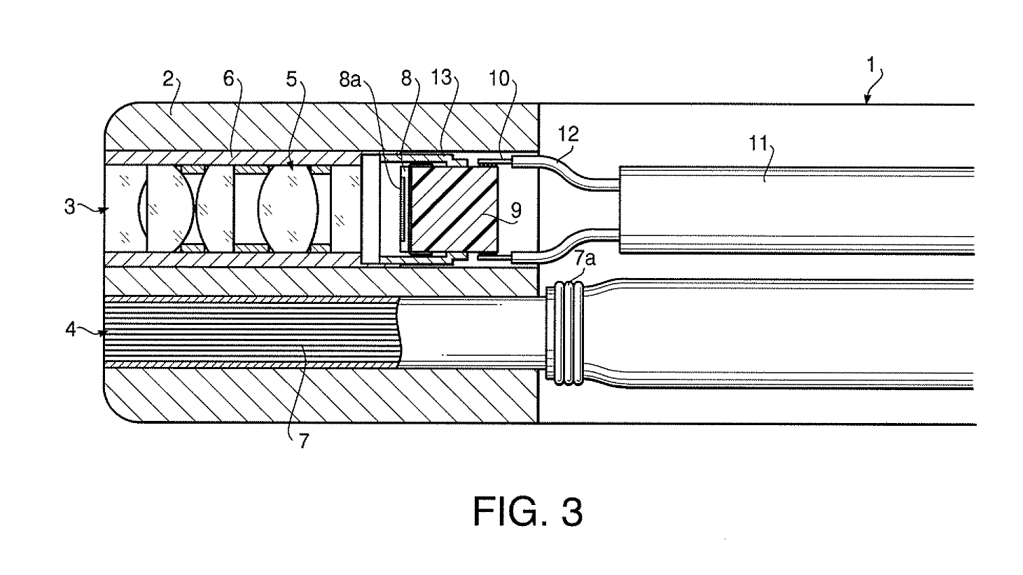

FIG. 3 shows a cross section of an inserting section 1 of an electronic endoscope. The distal end (i.e., the left-hand side end) of the inserting section 1 is connected to a tip body 2. On a distal end surface (i.e., the left-hand side end surface) of the tip body 2, an observation window 3 and an illumination window 4 are formed next to each other.

On an inner side of the observation window 3, an objective optical system 5 is provided. The objective optical system 5 includes multiple lenses, which are fixed inside an objective lens barrel 6, which has a cylindrical barrel. On an inner side of the illumination window 4, a light emitting end of an illumination light guide fiber bundle 7. In FIG. 3, 11 denotes a signal cable which is inserted through an entire length of the inserting section 1.

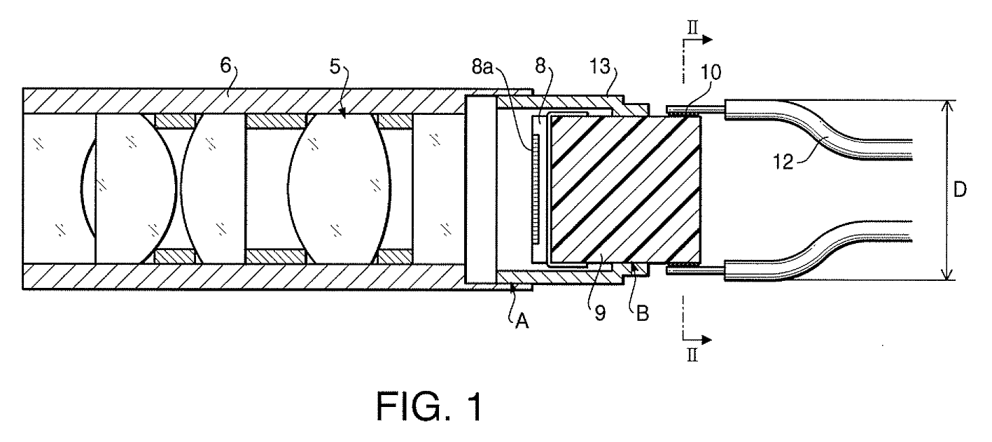

FIG. 1 shows an enlarged cross-sectional view of the objective optical system 5. On an image plane of the objective optical system 5, an image capturing surface 8a of a solid-state image capturing element 8 is located. The solid-state image capturing element 8 is attached to a front surface of an image capturing element frame 9, which is a substantially parallelepiped solid shape block made of electrically insulating material such as ceramic. Inside the image capturing element frame 9, an electronic circuit is provided and electronic parts such as an IC are implemented.

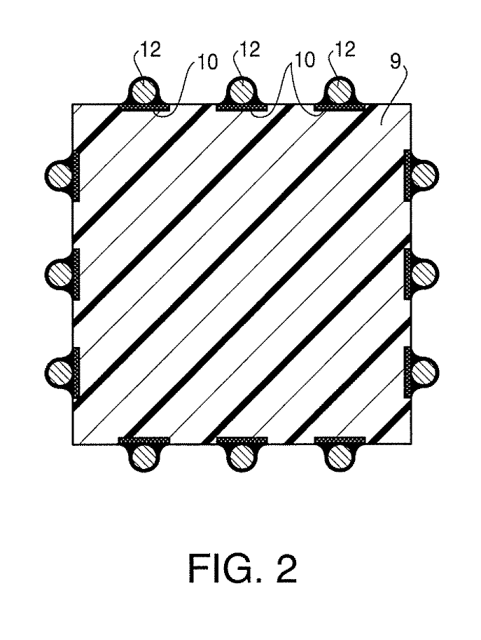

FIG. 2 is a cross section taken along line II-II of FIG. 1. The image capturing element frame 9 has a substantially square cross section and provided with multiple connection lands 10, which are arranged on a circumference at a rear portion of the image capturing element frame 9. To the connection lands 10, multiple signal lines 12 which extend at the tip end of the signal cable 11 are connected, for example, by soldering, respectively. It is noted that, throughout this specification, a term “circumference” is used to indicate a surface of a tube in a direction perpendicular to an axial direction of the tube, regardless of whether the cross sectional shape of the tube is circular or rectangular.

In FIG. 1 (also shown in FIG. 3), 13 denotes a metallic air-tight connection tube. The front side end (i.e., the left-hand side end) of the connection tube 13 is air-tightly connected to the objective lens barrel 6, and the rear side end (i.e., the right-hand side end) of the connection tube 13 is fixedly connected to the image capturing element frame 9. In this embodiment, the connection tube 13 is made of alloy containing Nickel, such as Kobar or 42 Alloy, having thermal expansion ratio substantially same as that of the image capturing element frame 9.

The front end portion of the air-tight connection tube 13 is fitted in an opening formed on a rear side end of the objective lens barrel 6, and fixed thereto air-tightly by welding or soldering (air-tight connected portion A). The rear side end of the air-tight connection tube 13 is fitted on the outer circumference of the image capturing element holding frame 9 at an intermediate position in axial direction (i.e., at a position on the front side of the connection lands 10), and fixed air-tightly by welding or soldering. With the above-described configuration, even in the high-temperature high-pressure steam sterilizer, the steam or water will not enter through the air-tightly fixed portion (air-tight connected portion B).

As a result of the above-described configuration, the outer size D) of the multiple signal lines 12 respectively connected to the connection lands 10 can be made smaller than the outer size of the objective lens barrel 6, and further, the outer size of the air-tight connection tube 13. Therefore, as shown in FIG. 3 for example, it is possible to make the tip body 2 sufficiently shin without interfering with outer tube bondage thread 7a of the illumination light guide fiber bundle and the like. In addition, since the multiple connection lands 10 are not arranged on the air-tight connection tube 13, electrical insulation can be made relatively easily.

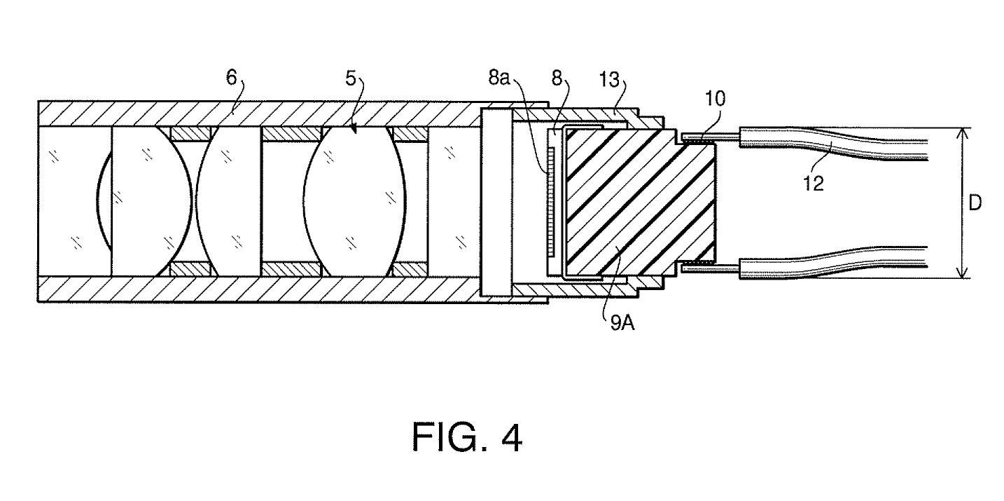

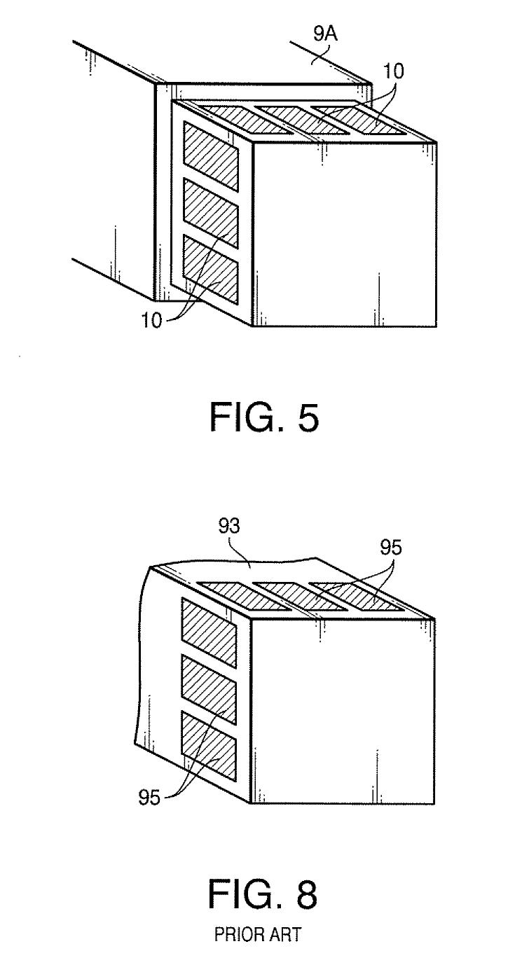

FIG. 4 shows a cross-sectional side view of the objective lens barrel 6 and the image capturing unit according to a second embodiment. FIG. 5 is a partial perspective view of a rear portion of an image capturing element holding frame 9A. According to the second embodiment, the outer size of the rear portion of the image capturing element holding frame 9A is smaller that the size of the front portion thereof (see FIGS. 4 and 5). With this configuration, the outer size D of the multiple signal lines 12 respectively connected to the multiple connection lands 10 can be smaller than that of the first embodiment.

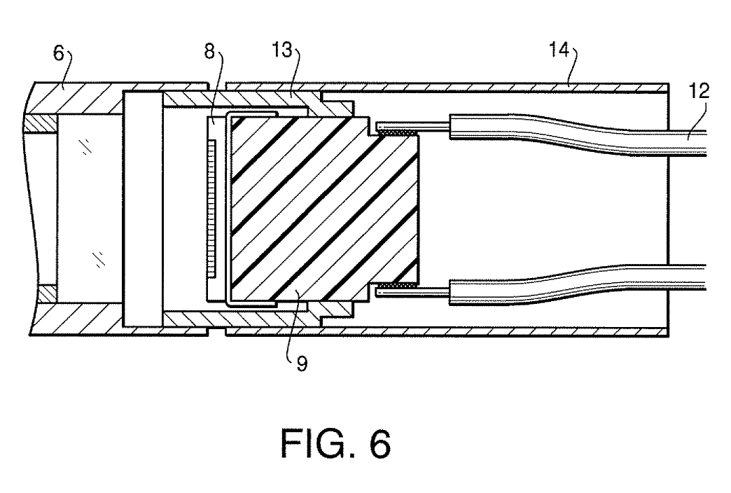

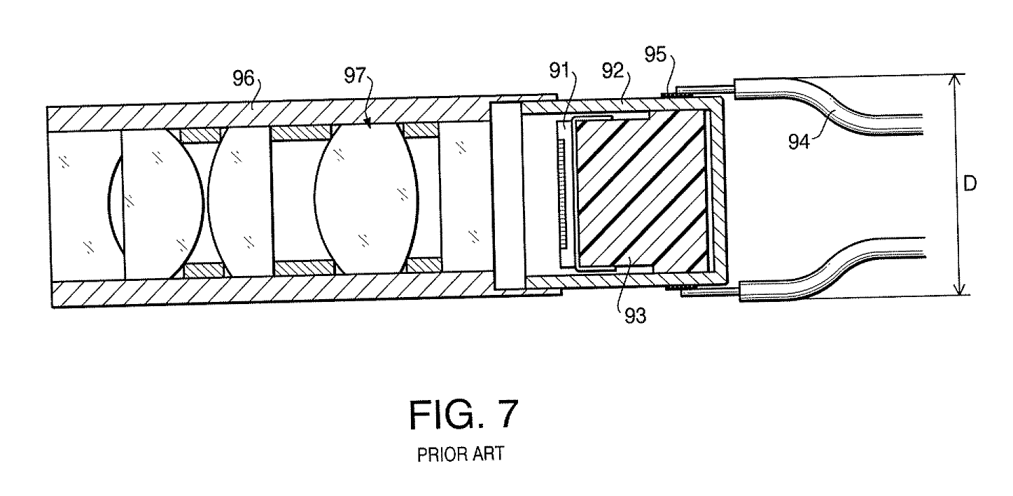

FIG. 6 shows a cross-sectional side view of the objective lens barrel 6 and the image capturing unit according to a third embodiment. In the third embodiment, in addition to the structure according to the second embodiment, an electrically conductive shielding tube 14 is provided to surround the multiple connection lands 10 and tip portions of the multiple signal lines 12 connected to the multiple connection lands 10, respectively. A front side portion of the shielding tube 14 is fitted on outer circumference of the air-tight connection tube 13 at an intermediate portion in the axial direction, and fixed thereto by welding or soldering.

With the above-described structure, the outer shape of the shielding tube 14 is not larger than the outer shape of the objective lens barrel 6. Therefore, according to the third embodiment, although the shielding tube 14 is provided, the electronic endoscope can be made sufficiently small.

The present disclosure relates to the subject matter contained in Japanese Patent Application No. 2005-201113, filed on Jul. 11, 2005, which is expressly incorporated herein by reference in its entirety.

What is claimed is:

1. An image capturing device for an electronic endoscope, comprising:
a solid-state image capturing element;
an image capturing element holding frame;
an objective lens barrel containing an objective optical system;
an air-tight connection tube, a front end portion of the connection tube being air-tightly connected to the objective lens barrel, a rear end portion of the connection tube being air-tightly fitted on the image capturing element holding frame,
wherein multiple connection lands to which multiple signal lines of a signal cable are connected are arranged on an outer circumferential surface, at a rear portion, of the image capturing element holding frame, the rear end portion of the connection tube being air-tightly connected to the outer circumference of the image capturing element holding frame on an front side with respect to the rear portion where the connection lands are arranged, a size of an outline of the multiple signal lines being smaller than an outer shape of the connection tube.

2. The image capturing device according to claim 1,
further comprising a conductive shielding tube configured to surround the multiple connection lands, an outer shape of the shielding tube in an axial direction thereof is smaller than an outer shape of the objective lens barrel in the axial direction of the shielding tube, a tip end portion of the shielding tube being fitted on an outer circumference of the air-tight connection tube.

3. The image capturing device according to claim 1,
wherein an outer shape of the image capturing element holding frame at the rear portion where the multiple connection lands are arranged is formed smaller than an outer shape of the front portion where the air-tight connection tube is secured.

4. The image capturing device according to claim 3,
further comprising a conductive shielding tube configured to surround the multiple connection lands, an outer shape of the shielding tube in an axial direction thereof is smaller than an outer shape of the objective lens barrel in the axial direction of the shielding tube, a tip end portion of the shielding tube being fitted on an outer circumference of the air-tight connection tube.
